(19) United States
(12) Patent Application Publication
(10) Pub. No.: US 2003/0124022 A1
(43) Pub. Date: Jul. 3, 2003

(54) Aroma dispensing wall plate

(76) Inventors: Chris Georges, Asbury Park, NY (US); Thomas Georges, Freehold, NJ (US)

Correspondence Address: William J. Sapone, Esq., Coleman Sudol Sapone P.C., 714 Colorado Ave., Bridgeport, CT, 06605, US

(21) Appl. No.: 10/189,022

(22) Filed: Jul. 2, 2002

Related U.S. Application Data
(60) Provisional application No. 60/381,577, filed on May 17, 2002.
(60) Provisional application No. 60/343,890, filed on Dec. 27, 2001.

(51) Int. Cl. A61L 9/00

(52) U.S. Cl. 422/5; 222/1; 422/4; 422/105

(57) ABSTRACT

An integral electrical connection box cover plate and air freshener has a chamber for receiving an aroma charge, a power heating circuit adjacent to the chamber and fittings for attachment to an electrical connection box. Contacts are provided with the back of the cover plate which engage the power circuit within the box such that one can provide an electrically powered air freshening device wherever an electrical connection box is located without using an electrical outlet.

### TECHNICAL FIELD

This invention relates to an electrically activated air freshener and more particularly to an integrated electrical connection box cover plate usable as an air freshening unit.

### BACKGROUND

Room air fresheners that utilize electrical power for releasing an air freshener are known. For example, in U.S. Pat. No. 6,104,866 an electrically heated chemical delivery system has a housing within which a heat activated chemical pouch is disposed. A circuit board with a heating element is located in the housing, with the housing having means for plugging into an outlet receptacle. When the housing is plugged into the receptacle, the chemical pouch is heated so that an aromatic gas is dispensed into the atmosphere.

In U.S. Pat. No. 6,302,559, a combined electo-luminescent night light and air freshener unit is described which similarly is plugged into a wall outlet so as to use electrical activation to thermally diffuse an aromatic gas into the surrounding environment, though in this case, the device can also function as a night light so as to use a single receptacle for both functions.

However, the problem remains that such devices are limited to receptacle locations and also require sacrificing an electrical outlet because of the continuous installation of the powered air freshener.

### SUMMARY OF THE INVENTION

It is an object of the present invention to provide an air freshening device that is electrically activated yet which does not need to be plugged into an electrical receptacle.

It is a further object of the present invention to provide an air freshening device that is electrically activated yet which can be located wherever an electrical connection box is located such as at switch locations, outlet locations or light fixture locations.

These and other objects of the present invention are achieved by an electrically actuatable air freshening device comprising:

a plate having means for mounting to an electrical connection box, the plate having inlet and outlet openings leading to a chamber integrated within the plate, a heat actuatable aromatizing agent removably disposed in the chamber, an electrically powered heat source integrated with a back of the plate and in communication with the chamber for delivering heat thereto, contact means, extending from the back of the plate for entering within the connection box for engaging electrical power leads therein. The plate may be a blank cover plate or be configured for use with various devices, having the appropriate openings for use with switches, single or duplex receptacles, lights, and combinations thereof, such that the plate functions as both a cover plate for the electrical connection box and as an air freshening device.

Using the present invention, air fresheners may be located wherever there is an electrical connection box, without requiring a plug-in connection and loss of an electrical outlet. The heating means is directly connected within the connection box to the power source, as an auxiliary connection to the connection made to the primary device held or retained within the electrical connection box, such as a switch or outlet, and thus utilization of the present invention does not reduce the number of receptacles in a room.

In one embodiment, when put into use, the replaceable air freshening charge is slid into and out of the chamber through a slot in the plate and is easily replaced when consumed or when a different fragrance is desired. As all of the other components of the air freshener are permanently installed with the cover plate, there is no needless replacement of the electrical components, reducing the lifetime cost of the air freshener. This is of particular advantage over the disposable plug in units presently used. As an option, a limit switch can be incorporated into the chamber such that heating only occurs when an air freshening charge is in place. Another option is a manual on/off switch, with or without variable control of the heating element to adjust the rate of diffusion of the aromatized product into the room. Yet another option is to integrate the heating element to the output of a light switch, if one is used in the box, such that the aroma is dispensed only when the light switch is activated.

Depending on the use of the particular cover plate, such as with an electrical switch or receptacle, the chamber and charge can be appropriately shaped so as to retain the slim plate profile to the extent possible. The chamber and charge are suitably shaped so as to be off-set from the device, or possibly surround the device to avoid interfering with its primary function.

Thus, the present invention increases the diversity of locations for placement of air fresheners, without consuming useable electrical receptacles. In fact, multiple placements are envisioned in individual rooms so as to increase the overall aromatic effect. With the invention, having multiple locations within a room allows using somewhat reduced volatility or strength aroma charges. This creates a more subtle aroma dispensing effect, which is more pleasing to the user, and avoids the somewhat over-powering effect that sometimes occurs with single point air freshening devices.

### BRIEF DESCRIPTION OF THE DRAWINGS

FIG. 1 is an exploded perspective of the components utilized in one embodiment of the present invention.

FIG. 2 is a view of a connection box with separate side terminals, FIG. 2a showing a companion cover plate.

FIG. 3 is a rear view of one embodiment of a cover plate produced in accordance with the present invention.

FIG. 4 is a side view thereof.

FIG. 5 is an exploded view of an alternative embodiment of the invention.

FIG. 6 is a view of yet another alternative embodiment of the invention.

FIG. 7 is rear view of the insert cover plate shown in FIG. 6.

FIG. 8 is a rear view of a holding plate showing snap in conductor leads for use with a single pole switch.

### DETAILED DESCRIPTION OF THE INVENTION

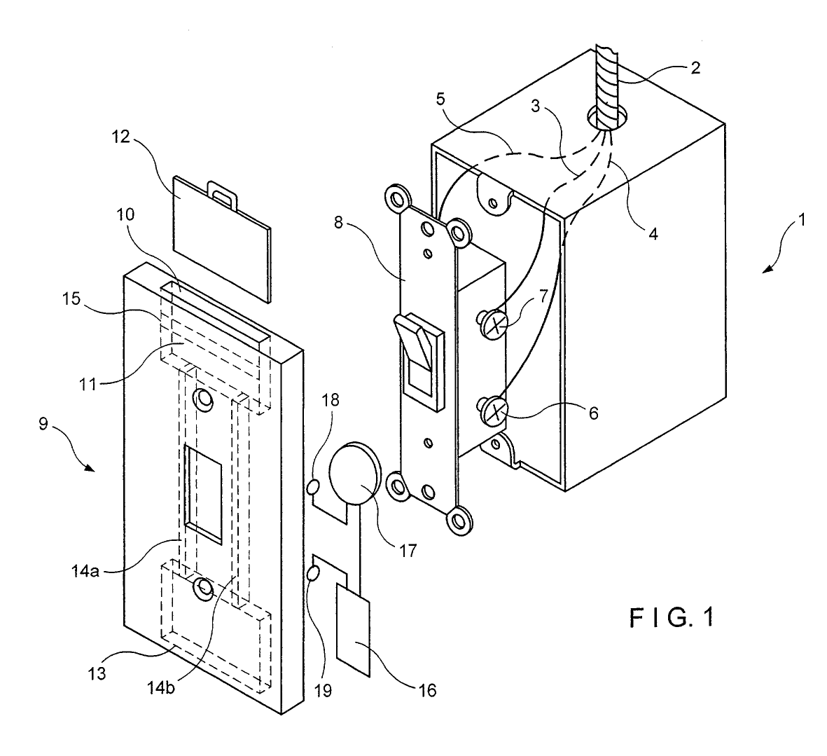

Referring to FIG. 1, the typical components utilized in and with the present invention are shown. The basic housing for the inventive device is a typical conventional electrical connection box 1 such as are used in homes, offices and other locations. These boxes typically have one or more cables 2 which enter through appropriate fittings. A basic cable typically contains three leads, a pair of power leads 3 and 4 and a copper grounding wire 5. The power leads are typically connected to a pair of contacts 6 and 7 on, for example, an electrical switch 8. Alternatively, jumpers can be used to connect the power leads to the contacts, such as when the cable is part of a chain of boxes on the same electrical circuit. Once a device such as the electrical switch is incorporated into the electrical connection box, a cover plate is applied.

FIG. 1 shows a cover plate 9 in accordance with the present invention. In this case, the cover plate 9 has a slot 10 on a top surface thereof which leads to a chamber 11 for containing an aroma charge 12 which may be slid into or out of the chamber by the user. The plate also has a bottom air inlet slot 13 with a pair of passages 14a and 14b leading to the chamber and also an air outlet 15, such that air can circulate through the chamber. A multi-slot or louver construction may also be used, integral with a front surface of the plate to promote air flow. Integrally mounted on the plate are a power control circuit 16 connected to a heating element 17. The heating element is disposed in proximity to the chamber so as to provide heating of the aroma charge. The power circuit has a pair of contacts 18 and 19 which in this case are prongs which extend from the back of the plate and are biased into engagement with the pair of contacts 6 and 7 on a side of the electrical switch such that no fixed connection is necessary to operate the unit. In other words, there is no hard wired connection between the power control heating circuit and the switch such that the plate can be added or removed without rewiring. Simply removing the cover plate breaks the circuit, allowing standard access to the elements in the box 1.

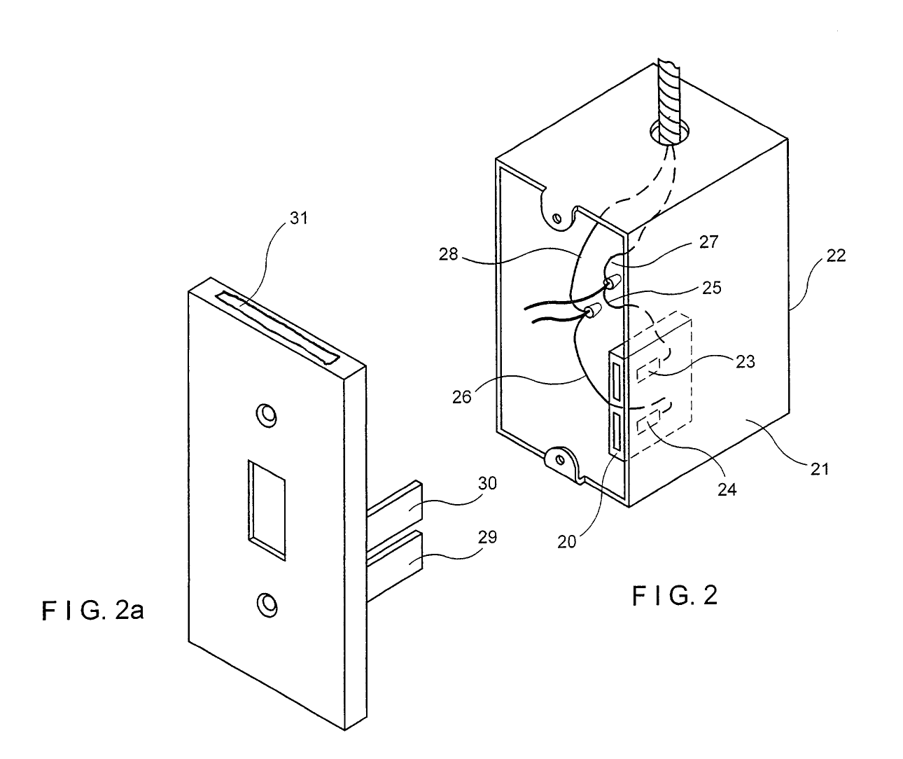

FIG. 2 shows an alternative embodiment of the invention. A receptor slot 20 is mounted to an inside side wall 21 of an electrical connector box 22, having a pair of contacts 23 15 and 24 connected by jumpers 25 and 26 to a pair of power supply leads 27 and 28. These are thus hard wired to the power leads. The slot contains the pair of contacts for slidingly receiving a pair of prongs 29 and 30 extending from the back of a plate 31, (see FIG. 2a) again to give a slide contact connection for the plate power circuit. This has the advantage of separating the electrical connection for the air freshener from the primary device to be mounted in the connection box, as on some occasions no primary device is used. This arrangement is adaptable to use with all manner of electrical components housed within the box, and can be fitted into multi-gang boxes as well. As in the previous embodiment, the wall plate can be added or removed without any additional hard wiring of the device.

All the electrical components and connections are internal to the electrical connection box and isolated by the plate wall. The plate is thus properly electrically isolated to provide a safe yet simple installation.

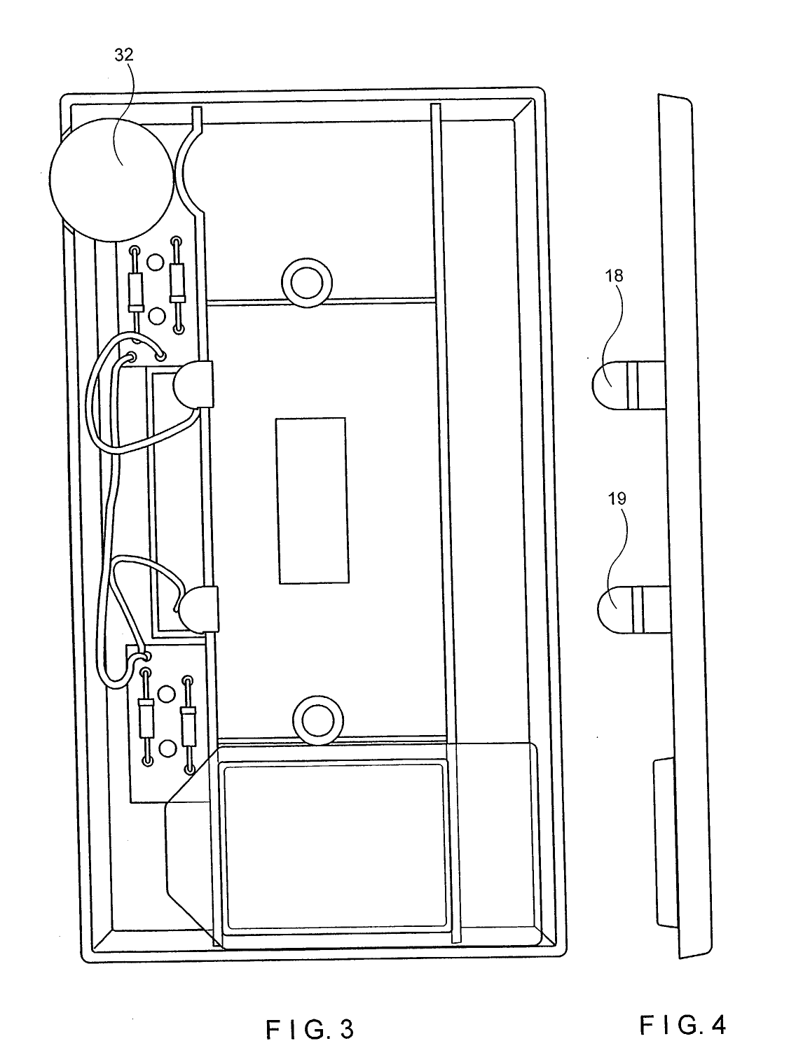

Referring to FIG. 3, a back view of an exemplary wall plate is shown. In this embodiment, there is included an optional thumb switch 32 which is part of the power circuit for on/off and/or variably controlling the heat applied to the aroma charge so as to shut off or vary the amount of aroma gas generated by the unit. In this plate, the aroma charge is disposed in a lower portion of the plate and may be slid in from a side of the plate instead of from the top. Various other arrangements are of course possible. FIG. 4 is a side view thereof.

The invention thus comprises an electrical wall plate which incorporates an air freshener in a single unit. The unit functions as an electrical connection box cover plate. While the box could be empty, such as when acting as a junction box, the box could contain a wall switch, outlet or another electrical device which allows these electrical devices to be used without interfering with the aroma generating function of the air freshener. The inventive wall plate is able to dispense aromatic gas at low operating temperatures, is energy efficient and compact and has a particular advantage in being a space saving design which increases the opportunities for placement of air fresheners within a room, without reducing available electrical outlets.

The combination wall plate and air freshener are manufactured from plastic, wood, metal, ceramic, or any material that commonly serves as a cover plate for an electrical connection box with plastic preferred. The air freshener cartridge is designed to contain a chemical aromatizing agent which is readily dissipated with or without heating, and this may comprise a fragrant gel, oil or another material, many of which are known in the art. Of course, with heating, the aromatic material is more readily dispensed into the atmosphere.

Various means may be used to provide electrical contact between the heating circuit and the power source in the electrical connection box. As discussed above, two electrical contacts may protrude from the rear of the plate inline with terminal screws on the electrical device located within the connection box. Alternatively, prongs may be incorporated with the plate for engagement with a pair of receptacles that can be disposed within the connection box along the side wall such that a sliding engagement of the plate completes the contact and provides power for the device, as shown in FIG. 2 and 2a. Of course, the invention also allows for the option of having direct hard wire connection to the power leads disposed within the electrical connection box, as will be discussed below.

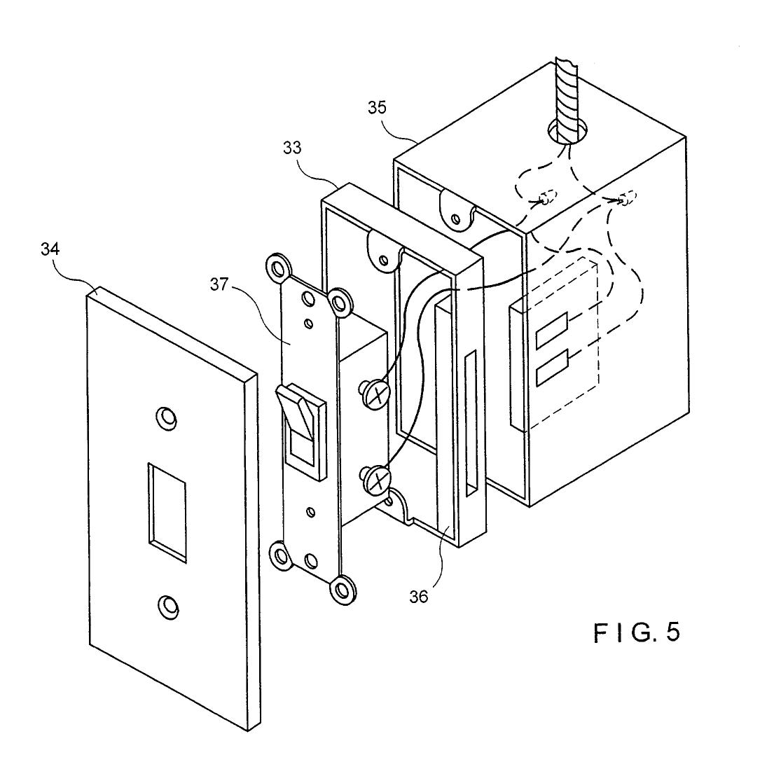

Another alternative embodiment of the invention incorporates a plate extension usable with a conventional wall plate, so as to enable a retrofitting to existing wall box installations, while using a conventional cover plate. Referring to FIG. 5, a plate extension 33 is shown which fits between a wall plate 34 and an electrical connection box 35. The extension includes a chamber 36 for receiving an aroma charge, and contains the power circuit and heating element as described above. The extension mounts directly to the wall box using conventional fittings, having threaded openings duplicating those found on the electrical box such that various electrical devices, are mountable to the extension such as the switch 37 shown. Of course, other means to fix the extension plate to the box may be used. While exaggerated for purposes of illustration, the extension is still of relatively thin profile and so does not significantly degrade the aesthetics of the wall plate. Once installed, with or without an electrical device, the conventional cover plate is attached. This has an advantage of enabling somewhat larger aroma charges to be used due to the increased area and/or thickness, and increases the ability to integrate larger flow through passages for dispensing the aroma gas into the room. All the options discussed above relative to the earlier embodiments are adaptable to this embodiment as well.

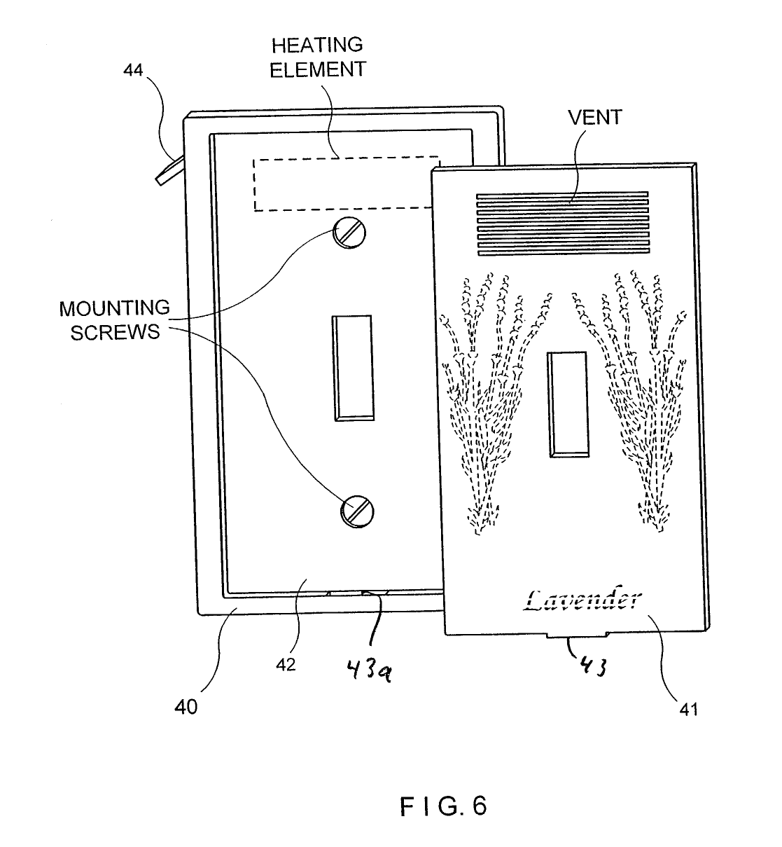

Referring to FIG. 6, another embodiment of the invention is shown. In this embodiment a base cover plate 40 is mountable to the electrical box as would be a conventional cover plate. However, the base cover plate has an insert 41 fitted into a recess 42 on the base plate, using a tab 43 that fits in a slot 43a. The insert is an integral unit containing the aroma charge, and vents. This is an alternative method of placing the charge on the base plate, and eases replacing the charge to alter the scent. Also, the insert itself can be produced of a plastic having a fragrance integral therewith. For example, POLYIFF® plastic resin is a matrix resin containing a mixture of scents that can be blended with other plastics to produce an aroma releasing plastic article. Thus, the insert itself can contain the aroma charge. POLYIFF® resin is available from International Flavor and Fragrance, Inc., Hazlet, N.J.

As described previously, the base plate 40 will contain the electrical components such as the heating element and the optional switch 44 for controlling the heating element. This embodiment offers more surface area and can hold a larger aroma charge and thus can increase the life of the aroma dispenser. It also allows for hard wiring of the power circuit on the base plate as the base cover plate is designed to remain in place for a long period of time. Using a flat insert also has the advantage of minimized packaging for replacement inserts, and again is cost effective as the powered components are reused.

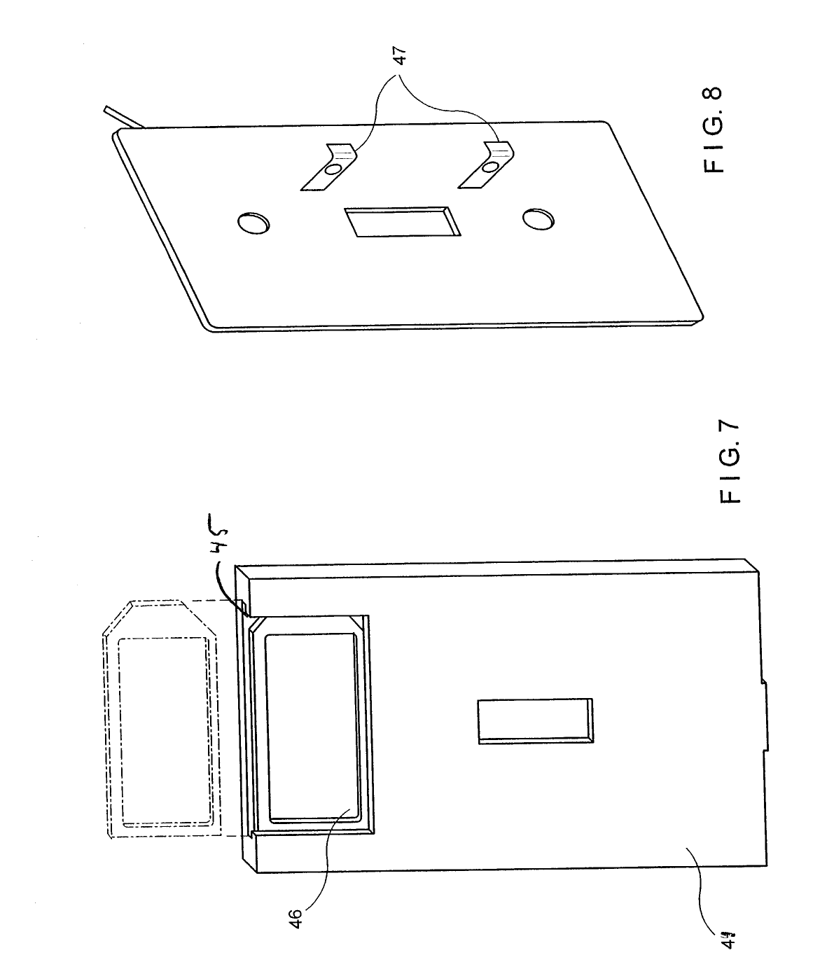

Referring to FIG. 7, a rear view of an insert 41 for the cover base plate 40 for mounting in a chamber 45 internally a replacement cartridge 46 is shown. FIG. 8 shows a rear view of a pair of resilient contacts 47 for engaging the terminals on a single pole electrical switch.

While preferred embodiments of the present invention have been shown and described, it will be understood that various changes or modifications could be made without varying from the scope of the present invention. For example, the invention is not limited in terms of the choice of aromatizing agents, as there are many commercially available agents as well as others that may become available in the future, and all these could be used with the present device, nor is the inventor limited in terms of cover plate dimensions or location of the aroma charge as one is free to adjust these to optimize aesthetics and functionality.

We claim:

1. An electrically actuatable air freshening device comprising a plate having means for mounting to an electrical connection box, the plate having openings leading to a chamber integrated therein, a heat activatable aromatizing agent removably disposed in the chamber, an electrically powered heat source integrated with the plate and in communication with the chamber for delivering heat thereto and contact means for connecting the heat source to a power source in the connection box.

2. The plate of claim 1 wherein slots are disposed in the plate so as to allow air to circulate therethrough.

3. The plate of claim 1 wherein the plate has one or more openings for receiving portions of an electrical component such that the plate functions as a cover plate for fitting over the electrical component and as an air freshening device.

4. The plate of claim 1 wherein the plate is composed of a material selected from the group consisting of plastic, metal, wood, and ceramic.

5. The plate of claim 1 wherein the contact means comprise at least one prong extending from a back of the plate for engagement with the power source in the connection box.

6. The plate of claim 1 wherein the contact means comprise at least one fitting for mating with a power lead from the power supply.

7. The plate of claim 1 wherein the contact means comprise at least one resilient prong extending from a back of the plate for engagement with the power source in the connection box.

8. The plate of claim 1 further comprising switch means associated with the electrically powered heat source.

9. The plate of claim 8 wherein the switch means are manually activated.

10. The plate of claim 8 wherein the switch means are associated with an electrical component located in the connection box and co-activated therewith.

11. The plate of claim 8 wherein the switch means provide on/off control.

12. The plate of claim 8 wherein the switch means provide variable control of the electrically powered heat source.

13. The plate of claim 3 wherein the electrical component is a toggle switch, the plate having an opening for the toggle to pass therethrough.

14. An electrically actuable air freshening device comprising an extension plate having means for mounting to an electrical connection box, the extension plate having openings leading to a chamber integrated therein, a heat activatable aromatizing agent removably disposed in the chamber, an electrically powered heat source integrated with the extension plate and in communication with the chamber for delivering heat thereto and contact means for connecting the heat source to power leads in the connection box, the extension plate having means for mounting an electrical device thereto such that a wall plate can be placed thereover.

15. The plate of claim 14 wherein slots are disposed in the plate so as to allow air to circulate therethrough.

16. The plate of claim 14 wherein the plate has one or more openings for receiving portions of an electrical component such that the plate functions as a cover plate for fitting over the electrical component and as an air freshening device.

17. The plate of claim 14 wherein the plate is composed of a material selected from the group consisting of plastic, metal, wood, and ceramic.

18. The plate of claim 14 wherein the contact means comprise at least one prong extending from a back of the plate for engagement with the power source in the connection box.

19. The plate of claim 14 wherein the contact means comprise at least one fitting for mating with a power lead from the power supply.

20. The plate of claim 14 wherein the contact means comprise at least one resilient prong extending from a back of the plate for engagement with the power source in the connection box.

21. The plate of claim 14 further comprising switch means associated with the electrically powered heat source.

22. The plate of claim 21 wherein the switch means are manually activated.

23. The plate of claim 21 wherein the switch means are associated with an electrical component located in the connection box and co-activated therewith.

24. The plate of claim 21 wherein the switch means provide on/off control.

25. The plate of claim 21 wherein the switch means provide variable control of the electrically powered heat source.

26. The plate of claim 16 wherein the electrical component is a toggle switch, the plate having an opening for the toggle to pass therethrough.

27. An electrically actuatable air freshening device comprising a base plate having means for mounting to an electrical connection box, an electrically powered heat source integrated with the plate for delivering heat thereto, the base plate having a recess on a front surface thereof, an aromatizing insert removably fitted to the recess and having an aroma charge associated therewith.

28. The plate of claim 27 wherein slots are disposed in the plate so as to allow air to circulate therethrough.

29. The plate of claim 27 wherein the plate has one or more openings for receiving portions of an electrical component such that the plate functions as a cover plate for fitting over the electrical component and as an air freshening device.

30. The plate of claim 27 wherein the plate is composed of a material selected from the group consisting of plastic, metal, wood, and ceramic.

31. The plate of claim 27 wherein the contact means comprise at least one prong extending from a back of the plate for engagement with the power source in the connection box.

32. The plate of claim 27 wherein the contact means comprise at least one fitting for mating with a power lead from the power supply.

33. The plate of claim 27 wherein the contact means comprise at least one resilient prong extending from a back of the plate for engagement with the power source in the connection box.

34. The plate of claim 27 further comprising switch means associated with the electrically powered heat source.

35. The plate of claim 34 wherein the switch means are manually activated.

36. The plate of claim 34 wherein the switch means are associated with an electrical component located in the connection box and co-activated therewith.

37. The plate of claim 34 wherein the switch means provide on/off control.

38. The plate of claim 34 wherein the switch means provide variable control of the electrically powered heat source.

39. The plate of claim 29 wherein the electrical component is a toggle switch, the plate having an opening for the toggle to pass therethrough.

40. A method for distributing an air freshening fragrance in a room having electrical connection boxes associated therewith comprising:
providing an electrically actuatable air freshening device comprising a plate having means for mounting to an electrical connection box, the plate having openings leading to a chamber integrated therein, a heat activatable aromatizing agent removably disposed in the chamber, an electrically powered heat source integrated with the plate and in communication with the chamber for delivering heat thereto and contact means for connecting the heat source to a power source in the connection box;
placing one or more electronically actuatable air freshening devices on one or more electrical connection boxes; and placing a heat actuatable aromatizing agent in the one or more freshening devices.
